(19) United States
(12) Patent Application Publication
(10) Pub. No.: US 2005/0148509 A1
(43) Pub. Date: Jul. 7, 2005

(54) Binding proteins as chemotherapy

(75) Inventors: Brian Dake, Mount Vernon, IA (US); Barbara A. Booth, Iowa City, IA (US); Mary Boes, Iowa City, IA (US); Robert S. Bar, Iowa City, IA (US)

Correspondence Address: Steven L. Highlander;FULBRIGHT & JAWORSKI L.L.P., Suite 2400, 600 Congress Avenue, Austin, TX, 78701, US

(73) Assignees: The University of Iowa Research Foundation; The US of America as represented by the Secretary of the Department of Veterans Affairs

(21) Appl. No.: 10/887,229

(22) Filed: Jul. 8, 2004

Related U.S. Application Data
(60) Provisional application No. 60/485,846, filed on Jul. 9, 2003.
(60) Provisional application No. 60/538,000, filed on Jan. 21, 2004.

(51) Int. Cl. A61K 38/18; C12Q 1/68; C07H 21/04; C07K 14/475; A61K 31/7072; A61K 31/704; A61K 31/4745; A61K 31/522

(52) U.S. Cl. 514/12; 514/34; 514/263.31; 514/284; 514/283; 514/575; 514/49; 514/255.02; 424/649; 435/6; 435/69.1; 435/320.1; 435/325; 530/399; 536/23.5; 514/251; 514/449; 514/109

(57) ABSTRACT

Embodiments of the invention include using molecular biology techniques to produce a fusion or chimeric protein of two or more different IGFBPs For example, the IGF binding properties or characteristics of a first protein are retained as is the receptor binding properties and characteristics of a second IGFBP. In particular aspects, a fusion or chimera of IGFBP-6 and IGFBP-3 (IGFBP-6/3) may be produced and used. Certain properties of each binding protein contribute to the therapeutic property of the fusion protein. For IGFBP-3 these properties include the specific binding to a target cell and the induction of apoptosis in a target cell. For IGFBP-6, these properties include an increased affinity for IGF-II versus IGF-I. The binding of IGF-II can deprive a cell exhibiting aberrant growth characteristics of the growth promoting functions of IGF-II.

This application claims priority to U.S. Provisional Patent Application Ser. No. 60/485,846, filed Jul. 9, 2003; and U.S. Provisional Patent Application Ser. No. 60/538,000 filed Jan. 21, 2004, which are incorporated in their entirety by reference.

The government owns rights in the present invention pursuant to grant number DK25421 and DK25295 from the National Institute of Diabetes and Digestive and Kidney Diseases. Mary Boes and Robert S. Bar are appointees of the Veterans Administration Medical Center, Iowa City, Iowa.

### BACKGROUND OF THE INVENTION

I. Field of the Invention

The present invention relates generally to the fields of molecular biology and oncology. More particularly, it concerns chimeric and fusion proteins derived from various members of the insulin-like growth factor binding proteins (IGFBPs).

II. Description of Related Art

The insulin-like growth factor (IGF) family of high affinity IGF binding proteins (IGFBP-1-IGFBP-6) (Lamson et al., 1991; Cohen and Rosenfeld, 1994; Jones and Clemmons, 1995; Rajram et al., 1997) has recently evolved to a superfamily status (Hwa et al., 1999). The conventional view of IGFBPs as the sole regulators of IGF bioavailability and bioactivity has also evolved to include the IGF-independent properties of IGFBPs (Kelley et al., 1996; Ferry et al., 1999). IGFBPs, particularly IGFBP-3, have been recently identified as potent apoptotic agents (Rechler, 1997; Valentinis et al.,. 1995; Zadeh and Binoux, 1997; Rajah et al., 1997; Oh, 1998), presumably mediating the effects of cellular growth suppressing mechanisms (Rechler, 1997; Zadeh and Binoux, 1997; Rajah et al., 1997; Oh, 1998). The emerging new concept appears to similarly broaden the pathophysiological roles of the IGF peptides to include their potential involvement in regulation of the IGFBPs' bioactivity (Rechler, 1997). In this ever-expanding maze of reciprocal molecular interactions, post-translational modification by selective proteolysis is rapidly gaining acceptance as the key modulator of the IGF/IGFBP system and a major determinant of their effects on cellular growth and metabolism (Rajah et al., 1995; Giudice, 1995).

Insulin-like growth factors (IGF-I and -II) are mitogenic and anti-apoptotic agents produced primarily by the liver and locally by a wide variety of tissues. IGFs circulate mostly complexed with IGFBP-3, which in association with the acid-labile subunit (ALS) forms an approximately 150 kD ternary protein complex (Lamson et al., 1991; Cohen and Rosenfeld, 1994; Jones and Clemmons, 1995; Rajram et al., 1997). Under normal conditions, nearly all of the circulating IGFs remain in a ternary complex (75-80%), and smaller proportions (20-25%) are associated with the low molecular weight IGFBPs (IGFBP-1, IGFBP-2, IGFBP-4, IGFBP-5, and IGFBP-6) or exist in the free form (Lamson et al., 1991; Cohen and Rosenfeld, 1994; Jones and Clemmons, 1995; Rajram et al., 1997).

Dysregulation and/or over-expression of the IGF system have been long implicated in the etiology of both benign and malignant proliferative disorders (Jones and Clemmons, 1995; Rajram et al., 1997; Russell et al., 1998; Holly, 1998; Rosen and Pollak, 1999; Cohen, 1998; Baserga, 1995). Malignant cells of various origins have been shown to express various components of the IGF system (Jones and Clemmons, 1995; Rajram et al., 1997; Oh, 1998; Rajah et al., 1995; Cohen, 1998; Baserga, 1995; Li et al., 1998; Glick et al., 1997; Lahm et al., 1994), and increased IGF-I levels, as seen in acromegaly, have been found in association with benign prostatic hyperplasia (BPH) (Grimberg and Cohen, 1999; Colao et al., 1999) and colonic tumors (Cats et al., 1996; Orme et al., 1998). High levels of circulating IGF-I has been more recently identified as risk factors for the development of prostate, breast, and lung cancers (Chan et al., 1998; Hankinson et al., 1998; Wolk et al., 1998; Yu et al., 1999), while over-expression of both IGF-I and IGF-II has been linked to colorectal cancers (Manousos et al., 1999). In prostate, both benign and malignant cells have been found to express IGFs, IGFBPs and their respective receptors (Cohen, 1998; Grimberg and Cohen, 1999). IGF-I has been shown to promote prostate cell growth, while prostate specific antigen (PSA) has been identified as an IGFBP-3 protease, presumably capable of augmenting tissue access to the IGF peptides (Cohen, 1998; Grimberg and Cohen, 1999; Cohen et al., 1992).

IGF-II has been implicated in the proliferation of several cancers, including neuroblastoma (Carlsen, 1992). Neuroblastoma is the second most common solid tumor in childhood with only cranial tumors being more prevalent. Some neuroblastomas spontaneously regress while others are more aggressive. When neuroblastomas reach stages III and IV, prognosis is poor with few therapeutic options.

### SUMMARY OF THE INVENTION

Thus, in accordance with the present invention, there is provided a recombinant fusion protein comprising a growth factor binding domain and a cell association domain, wherein the growth factor binding domain, and the cell association domain are operably linked. The domains may or may not be separated from each other. Separation of the domains may be by 1-20 amino acid residues. In certain embodiments, the growth factor binding domain and the cell association domain may not occur in a single non-recombinant protein.

Certain aspects of the invention include using molecular biology techniques to produce a fusion or chimeric protein of all or part of at least two proteins, e.g., IGFBPs, such that the growth factor binding domain of a first protein are retained and the receptor binding properties of a second protein is retain. In particular aspects, a fusion or chimera comprising an amino terminal IGFBP-6 or fragment thereof and a carboxy terminal IGFBP-3 (IGFBP-6/3) may be produced and used. IGFBP-3 provides, for example, the properties of specific binding to a target cell and induction of apoptosis in particular target cells. For IGFBP-6, these properties include an increased affinity for IGF-II versus IGF-I. The binding of IGF-II can deprive a cell exhibiting aberrant growth characteristics of the growth promoting functions of IGF-II.

In certain embodiments, a polypeptide may include a polypeptide comprising all or part of an amino acid sequence of a first IGFBP protein at an amino terminus of the polypeptide and all or part of an amino acid sequence of a second IGFBP at a carboxy terminus of the polypeptide. The polypeptide may include a growth factor binding domain and/or a cell association domain. A first IGFBP protein may include all or part of IGFBP-1, IGFBP-2, IGFBP-3, IGFBP-4, IGFBP-5 or IGFBP-6. In cetain embodiments the first IGFBP protein is IGFBP-6. The second IGFBP protein may include all or part of IGFBP-1, IGFBP-2, IGFBP-3, IGFBP-4, IGFBP-5 or IGFBP-6. In certain embodiments the second IGFBP is IGFBP-3. In other embodiments, the polypeptide the first IGFBP protein is all or part of IGFBP-6 and the second IGFBP protein is all or part of IGFBP-3. The polypeptide may be in a pharmaceutically acceptable formulation.

In other embodiments, a polynucleotide or nucleic acid includes a nucleic acid sequence that encodes a polypeptide comprising a fusion of a first IGFBP and a second IGFBP amino acid sequence is contemplated. The nucleic acid may include a promoter region, a polyadenylation signal, or other regulatory sequences known to one of ordinary skill in the art. The nucleic acid may include a first IGFBP and a second IGFBP. In some embodiments the first IGFBP can be IGFBP-1, IGFBP-2, IGFBP-3, IGFBP-4, IGFBP-5 or IGFBP-6. In particular embodiments, the first IGFBP is IGFBP-6. The second IGFBP can be IGFBP-1, IGFBP-2, IGFBP-3, IGFBP-4, IGFBP-5 or IGFBP-6. In particular embodiments the second IGFBP is IGFBP-3. In certain embodiments the first IGFBP is all or part of IGFBP-6 and the second IGFBP is all or part of IGFBP-3. In particular embodiments the nucleic acid is included in an expression vector.

Certain embodiments include methods of producing a polypeptide, as described above, comprising: (a) culturing a host cell comprising a poynucleotide encoding a polypeptide comprising all or part of an amino acid sequence of a first IGFBP protein at an amino terminus of the polypeptide and all or part of an amino acid sequence of a second IGFBP at a carboxy terminus of the polypeptide under conditions which allow for expression of the polypeptide; and (b) recovering the polypeptide from the cells. The polypeptide may include, as the first IGFBP protein, IGFBP-1, IGFBP-2, IGFBP-3, IGFBP-4, IGFBP-5 or IGFBP-6. In particular embodiments, the first IGFBP is IGFBP-6. The second IGFBP can be IGFBP-1, IGFBP-2, IGFBP-3, IGFBP-4, IGFBP-5 or IGFBP-6. In particular embodiments the second IGFBP is IGFBP-3. In certain embodiments the first IGFBP is all or part of IGFBP-6 and the second IGFBP is all or part of IGFBP-3.

Other embodiments include methods of treating a cancer patient or a patient suffering from a hyperproliferative disease comprising administering an effective amount of a polypeptide comprising all or part of an amino acid sequence of a first and a second IGFBP protein. The polypeptide may include, as the first IGFBP protein, IGFBP-1, IGFBP-2, IGFBP-3, IGFBP-4, IGFBP-5 or IGFBP-6. In particular embodiments, the first IGFBP is IGFBP-6. The second IGFBP can be IGFBP-1, IGFBP-2, IGFBP-3, IGFBP-4, IGFBP-5 or IGFBP-6. In particular embodiments the second IGFBP is IGFBP-3. In certain embodiments, the first IGFBP is all or part of IGFBP-6 and the second IGFBP is all or part of IGFBP-3. The method may further comprise administering at least a second anti-cancer therapeutic to the patient. The cancer may be a neuronal, prostate, lung, brain, skin, liver, breast, blood, stomach, testicular, ovarian, pancreatic, bone, bone marrow, head and neck, cervical, esophageal, gall bladder, kidney, adrenal, or colon rectal cancer. In particular embodiments, the cancer is a neuroblastoma or a rhabdomyosarcoma. The polypeptide may be administered in various ways known to one skilled in the art including, but not limited to intravenous, intradermal, intraarterial, intraperitoneal, intraarticular, intrapleural, intratracheal, intranasal, intravaginal, topical, intramuscular, subcutaneous, intravesicular, mucosal, oral, or aerosol administration.

In still further embodiments, the method may include administering at least a second, third, fourth, fifth or more anti-cancer therapeutic to the patient. The second, third, fourth, fifth, or more anti-cancer therapeutic may be an alkylating agent, topisomerase I inhibitor, topoisomerase II inhibitor, RNA/DNA antimetabolite, DNA antimetabolite, antimitotic agent, and DNA damaging agent. In certain aspects, the alkylating agent may be chloroambucil, cis-platinum, cyclodisone, flurodopan, methyl CCNU, piperazinedione, or teroxirone. The topisomerase I inhibitor may be camptothecin, camptothecin derivatives, or morpholinodoxorubicin. The topoisomerase II inhibitor may be doxorubicin, pyrazoloacridine, mitoxantrone, or rubidazone. The RNA/DNA antimetabolite may be L-alanosine, 5-fluoraouracil, aminopterin derivatives, methotrexate, or pyrazofurin. The DNA antimetabolite may be ara-C, guanozole, hydroxyurea, or thiopurine. The antimitotic agent may be colchicine, rhizoxin, taxol, or vinblastine sulfate. The DNA damaging agent may be γ-irradiation, X-rays, UV-irradiation, microwaves, electronic emissions, adriamycin, bleomycin, 5-fluorouracil (5FU), etoposide (VP-16), camptothecin, actinomycin-D, mitomycin C, cisplatin (CDDP), podophyllotoxin, verapamil, or hydrogen peroxide.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Shows a purified fusion protein of IGFBP-6 and IGFBP-3 (FP6/3) (20 μg) on 12% SDS-PAGE gel (left lane with Coomassie blue), ligand blot for fusion protein FP 6/3, IGFBP-3 and IGFBP-6 with ¹²⁵I-IGF-I or ¹²⁵I-IGF-II (center lanes) and immunoblots (right lanes) of the same IGFBPs using antiserum against IGFBP-3 or IGFBP-6. Molecular weight markers are shown on the left and location of IGFBPs designated by markers on the right.

FIG. 2A-2E. Shows binding of fusion protein to microvessel endothelial cells (FIG. 2A). Binding of ¹²⁵I-IGFBP-3 (□) or 125I-FP 6/3 (▪) to neuroblastoma cell lines SHSY-5Y (FIG. 2B), SK-N-SH (FIG. 2C) and rhabdomyosarcoma cell lines RD (FIG. 2D) and Rh 30 (FIG. 2E). Total binding is given with ¹²⁵I-IGFBP-3 and ¹²⁵I-FP 6/3 (20,000 cpm) versus unlabeled IGFBP-3, FP-6/3, IGFBP-6 (50 μg/ml). Incubation was for 90 min at 22° C. Data represent the mean±SEM of three separate wells. ***P<0.001 compared to total binding (ANOVA/Newman-Keuls).

FIG. 3A-3B. FIG. 3A shows the effect of concentration (1, 10, 100 nM) of FP 6/3, IGFBP-3, IGFBP-6 or IGFBP-3+6 on thymidine incorporation into DNA in SHSY-5Y neuroblastoma cells with coincubation of IGF-II (50 ng/ml) for 18 h. FIG. 3B shows the effect of transient exposure on SHSY-5Y neuroblastoma cells and subsequent inhibition of thymidine incorporation. Transient conditions were exposure to FP 6/3 or binding proteins (100 nM) for different times (10, 30, 60 min), removal and then stimulation with IGF-II (6.5 nM) for 18 h. Increased frequency condition (×2) was treatment at time 0 and then repeated at 9 h. Data represent the mean±SEM of three separate wells. ***P<0.001, **P<0.01, *P<0.05 compared to IGF-II (ANOVA/Newman Keuls).

FIG. 4A-4B. FIG. 4A shows the effect of FP 6/3, IGFBP-3, IGFBP-6 or IGFBP-3+6 (100 nM) on thymidine incorporation in SK-N-SH neuroblastoma cells with coincubation of IGF-II (6.5 nM) for 18 h. FIG. 4B shows the effect of transient exposure on SK-N-SH neuroblastoma cells and subsequent thymidine incorporation. Transient conditions were exposure for 30 min to binding proteins (100 nM) or fusion protein (1, 10, 100 nM), removal and then stimulation with IGF-II (6.5 nM ng/ml) for 18 h. FP 6/3 at 100 nM for 18 h+IGF-II shown for reference. Data represent the mean±SEM of three separate wells. ***P<0.001, **P<0.01, *P<0.05 compared to IGF-II (ANOVA/Newman Keuls).

FIG. 5: Shows the effect of concentration and transient exposure in RD rhabdomyosarcoma cells and subsequent thymidine incorporation. Transient conditions were exposure for 30 min to either binding proteins (100 nM) or FP 6/3 (1, 10, 50, 100 nM), removal and then stimulation with IGF-II (6.5 nM) for 18 h. FP 6/3 at 100 nM for 18 h with coincubation of IGF-II is shown for reference. Data represent the mean±SEM of three separate wells. ***P<0.001, **P<0.01, *P<0.05 compared to IGF-II (ANOVA/Newman Keuls).

### DESCRIPTION OF ILLUSTRATIVE EMBODIMENTS

Insulin-like growth factor-II (IGF-II) has been implicated in the proliferation of several cancers, including neuroblastoma (Carlsen, 1992). Neuroblastoma is the second most common solid tumor in childhood with only cranial tumors being more prevalent. Some neuroblastomas spontaneously regress while others are more aggressive. When neuroblastomas reach stages III and IV, prognosis is poor with few therapeutic options. Embodiments of the invention may be used to provide treatment for neuroblastoma and other cancers or hyperproliferative conditions.

Neuroblastomas are inhibited by agents which specifically target IGF-II, such as insulin-like growth factor binding protein-6 (IGFBP-6) (Grellier et al., 2002). IGFBP-6 is distinguished from the other five high affinity IGF binding proteins, IGFBP-1-IGFBP-5, by its affinity for IGF-II, which is 20-100 times greater than for IGF-I (Bach, 1999). Neuroblastoma cells derived from human neuroblastomas are growth inhibited when engineered to make IGFBP-6 (Seurin et al., 2002).

Embodiments of the invention include binding proteins that may function as a growth inhibitor, like IGFBP-6, but also possess an ability to bind to cells, like IGFBP-3. In particular embodiments, the binding protein may be a fusion protein (FP 6/3), or a proteinaceous molecule wherein all or part of IGFBP-6 and IGFBP-3 are operatively coupled, wherein the proteinaceous molecule binds to cancer cells and inhibits cellular proliferation.

Fusion proteins of the invention may comprise all or part of two or more of Insulin-like Growth Factor Binding Proteins (IGFBPs). In particular embodiments, an IGFBP fusion protein may be an IGFBP-6/IGFBP-3 (FP6/3) fusion protein. An FP6/3 fusion may include all or part of the IGFBP-6 polypeptide, wherein the polypeptide or fragment thereof imparts an IGF-II binding characteristic to the fusion protein. An FP6/3 fusion may include all or part of an IGFBP-3, wherein the poypeptide or peptide imparts the characteristic of binding to one or more cells to a fusion protein.

Binding proteins of the invention may be used in methods of treating or ameliorating a cancerous disease state.

I. Binding Peptides and Polypeptides

Embodiments of the invention provide fusion proteins for use in alleviating, inhibiting, or treating cancer or hyperproliferative disorders. The fusions typically comprise a first domain that is a growth factor binding domain and a second domain that is a cell association domain. The domains may or may not be separated by a spacer, for example 1 to 20 amino acid residues. The first and second domains may or may not occur in a single recombinant protein. In certain embodiments the amino terminus of one domain is fused to the carboxy terminus of a second domain.

It is contemplated that the fusion protein may further comprise at least two growth factor binding and/or cell association domains. These additional domains may be multiples of the same growth factor binding and/or cell association domain or may be different growth factor binding or cell association domains.

The term “separated by” refers to the recited number of residues present, if any, between the domains, thus separating the domains.

As used herein, “growth factor binding” means binding of a growth factor of interest to the binding domain. Binding may be by covalent or non-covalent interaction. Binding of the growth factor will typically reduce, diminish, or negate one or more growth promoting characteristic of the growth factor bound to the growth factor binding domain.

The term “cell association” refers to the preferential localization to the surface of a particular cell type or population of cells such as cells of a tumor or cells exhibiting a cancerous or hyperproliferative phenotype. The cell association domain will typically demonstrate an affinity for a particular feature of a cell surface such as receptors, structural proteins, glycoproteins, glycosylated surface components and the like.

In particular, the growth factor binding domain may have an affinity for an insulin-like growth factor. Insulin-like growth factor (IGF) action is influenced by the insulin-like growth factor binding proteins (IGFBP). The ability of IGFBPs to form complexes with IGFs influence the transport of IGFs to membrane receptors and modulate IGFs effects on cell proliferation. IGFBPs are proteins of different size which are produced by many different tissues and they bind to IGF-I, IGF-II, but not to insulin. The affinity constants of the six IGFBPs are similar for IGF-I and IGF-II (approximately 2-20 and 3-30×10⁹ l/mol, respectively), with the exception of IGFBP-6, which has an approxiamte 20- to 70-fold or higher affinity for IGF-II than for IGF-I (Zapf et al., 1995). IGFBP molecules typically contain 18 cysteine residues, six of them being located in carboxy terminus and twelve in amino terminus. IGFBPs modulate IGFs effects by endocrine, paracrine and autocrine mechanisms (Martin and Baxter, 1999).

Six structurally distinct insulin-like growth factor binding proteins, which have a high affinity for IGFs, have been isolated and their cDNAs cloned: IGFBP-1, IGFBP-2, IGFBP-3, IGFBP-4, IGFBP-5, and IGFBP-6. The proteins display strong sequence homologies. The IGFBPs contain 3 structurally distinct domains each comprising approximately one-third of the molecule. The N-terminal domain I and the C-terminal domain 3 of the 6 human IGFBPs show moderate to high levels of sequence identity including 12 and 6 invariant cysteine residues in domains 1 and 3, respectively (IGFBP-6 contains 10 cysteine residues in domain 1), and are thought to be the IGF binding domains. Domain 2 is defined primarily by a lack of sequence identity among the 6 IGFBPs and by a lack of cysteine residues, though it does contain 2 cysteines in IGFBP-4. Domain 3 is homologous to the thyroglobulin type I repeat unit. A review of the various IGFBP and their function may be found in Kostecka and Blahovec (1999). IGFBP 1-6 are described below.

IGFBP-1—IGFBP-1 is a 25-34 kDa protein that was originally isolated from human placenta. IGFBP-1 has also been identified in serum in 100-fold lower concentration than IGFBP-3. IGFBP-1 of amniotic fluid binds with both IGFs with high affinity: Kₐ=6.55±2.24 l/nmol for IGF-I and Kₐ=3.23±1.05 l/nmol for IGF-II (Baxter et al., 1987). IGFBP-1 is an acid-stable protein. It is not a placental protein in the true sense, but is secreted by the endometrium or the decidua.

IGFBP-1 inhibits both placental and fetal growth by minimizing the amount of IGF molecules available in the maternal organism. By binding and neutralizing free IGF, unlimited proliferation of the trophoblast into the decidual endometrium is prevented. High IGFBP-1 concentrations may lead to retardation, at the worst to the intrauterine death of the fetus and to miscarriage.

IGFBP-2—IGFBP-2 is a 32-34 kDa protein that has been found in cerebrospinal fluid, seminal plasma, lymph and other fluids sampled from various animals. IGFBP-2 contains a signal peptide and is secreted from many cells. Intact IGFBP-2 has comparably high affinity for the IGFs and acts as an inhibitor of IGF-I or -II. Proteolysis of IGFBP-2 gives rise to fragments characterized by reduced affinities for the IGFs and bound IGFs thus dissociate from IGFBP-2 and might trigger signal cascades via the IGF-I receptor. IGFBP-2 also binds to the extracellular matrix (ECM), integrin receptors (due to its RGD motif) or glycosaminoglycans (GAG).

IGFBP-3—IGFBP-3 is an approximately 40-45 kDa protein. Fraser et al. (2000) found that IGFBP-3 mRNA is expressed in the endothelium of the human corpus luteum and that the levels of message change during luteal development and rescue by human chorionic gonadotropin (CG). The signal was strong during the early luteal phase, but showed significant reduction during the mid- and late luteal phases. Administration of human CG caused a marked increase in the levels of IGFBP-3 mRNA in luteal endothelial cells that was comparable to that observed during the early luteal phase. The authors concluded that endothelial cell IGFBP-3 expression is a physiologic property of the corpus luteum of menstruation and pregnancy, and they speculated that the regulated expression of endothelial IGFBP-3 may play a role in controlling angiogenesis and cell responses in the human corpus luteum by autocrine/paracrine mechanisms.

Popovici et al. (2001) established highly pure primary cultures of human fetal hepatocytes in vitro and investigated the expression of IGFBP-1 and the effects of hypoxia on expression of IGFBP-1 mRNA and protein. Western blot analysis of conditioned medium revealed the presence of IGFBP-1, IGFBP-2, IGFBP-3, and IGFBP-4. A 3-fold increase in IGFBP-3 mRNA, but not other IGFBPs, was noted under hypoxic, compared with normoxic, conditions. The authors concluded that hypoxia upregulates fetal hepatocyte IGFBP-1 mRNA steady-state levels and protein, with this being the major IGFBP derived from the fetal hepatocyte.

Deal et al. (2001) pointed to evidence that the circulating level of IGFBP-3 is inversely related to the risk of several common cancers, and that antiproliferative agents such as antiestrogens and retinoids act in part by upregulating IGFBP-3 expression. It is believed that both growth-inhibitory and growth-potentiating effects of IGFBP-3 on cells are independent of IGF action and are mediated through specific IGFBP-3-binding proteins/receptors located at the cell membrane, cytosol, or nuclear compartments and in the extracellular matrix.

To examine more critically the amino acids important for IGF binding within the full-length IGFBP-3 protein while minimizing changes in the tertiary structure, Buckway et al. (2001) targeted residues I56, L80, and L81 within the proposed hydrophobic pocket for mutation. With a single change at these sites to the non-conserved glycine there was a notable decrease in binding. A greater reduction was seen when both L80 and L81 were substituted with glycine, and complete loss of affinity for IGF-I and IGF-II occurred when all 3 targeted amino acids were changed to glycine. The authors concluded that their data supported the hypothesis that an N-terminal hydrophobic pocket is the primary site of high affinity binding of IGF to IGFBP-3.

Spoerri et al. (2003) found that cultured human retinal endothelial cells expressed endogenous IGFBP-3. Exogenous administration of IGFBP-3 induced growth inhibition and apoptosis, supporting a regulatory role for IGFBP-3 in endothelial cells. Somatostatin receptor (SSTR) agonists mediated their growth-inhibitory effect, in part, by increasing expression of IGFBP-3.

IGFBP-4—IGFBP-4 is an approximately 24 kDa protein. The fifteen NH₂-terminal amino acids of IGFBP-4 are identical with those of IGFBP-5. The prepeptide sequences of BP-4 contains 27 amino acids and the mature protein contains 213 amino acids (Mr=22,610). The NH₂- and COOH-terminal thirds of BP-4 display pronounced homology to the other three human BPs. Sixteen of the 16-20 cysteines and 37 of the 213-289 amino acids (12.8-17.1%) are conserved in all IGFBs 1-5. Ten amino acid positions located in the NH₂-terminal region and shared by IGFBP-1, -2, -3, and -5 are different in IGFBP-4. These differences may account for the preferential affinity of IGFBP-4 for IGF II.

IGFBP-5—IGFBP-5 is an approximately 23 kDa protein. Allander et al. (1994) cloned the IGFBP-5 gene from a human genomic library and showed that it is divided into 4 exons which, primarily due to a first intron of approximately 25 kb, span about 33 kb of DNA. Southern analysis identified a single copy of the IGFBP-5 gene in the haploid human genome and is located on human 2q33-q34. The IGFBP-2 gene and the IGFBP-5 gene are transcribed convergently and are separated by approximately 20 to 40 kb of DNA.

IGFBP-6—IGFBP-6 is an approximately 30-32 kDa protein. Shimasaki et al. (1991) cloned the IGFBP-6 gene and showed that in the human it codes for a 216-amino acid protein with a calculated molecular weight of 22,847. A single 1.3-kb IGFBP-6 mRNA was detected by Northern analysis in all rat tissues examined, indicating that this binding protein is ubiquitous. Using PCR on human/hamster somatic cell hybrid DNAs, Shimasaki et al. (1991) determined that the IGFBP-6 gene is located on chromosome 12.

Kato et al. (1995) showed that the human keratinocyte cell line HaCat secretes IGFBP-6 as an autocrine growth inhibitor. Recombinant IGFBP-6 was also shown to inhibit growth of HaCat cells and other keratinocyte cell lines.

A. Fusion Proteins and Protein Variants

A fusion protein is a specialized type of insertional variant. This molecule generally has all or a substantial portion of a first molecule or polypeptide, linked at the N- or C-terminus, to all or a substantial portion of a second polypeptide. For example, fusions can employ leader sequences from other species to permit the recombinant expression of a protein in a heterologous host. Another useful fusion includes the addition of a region to facilitate purification of the fusion protein. Inclusion of a cleavage site at or near the fusion junction will facilitate removal of the extraneous polypeptide after purification. Other useful fusions include linking of functional domains, such as ligand-binding domains, e.g., an IGF-II binding domain; glycosylation domains; cellular targeting signals; transmembrane regions; or receptor-binding domains. For example, a fusion may comprise ligand-binding or growth factor binding domain and cell interaction or association domain for the localization of the growth factor binding function, e.g. FP6/3 SEQ ID NO:15 and 16.

As used herein, “fusion protein” means a non-naturally occurring protein product, wherein the domains of the fusion protein are derived from one or more other proteins or artificially derived sequences. For example, each domain can be derived from a different naturally occurring protein sequence, or mutant/variant thereof, that possesses the desired properties, e.g., IGFBP1-IGFBP-7; and/or the domains can be derived from a naturally occurring protein. Variations on this theme will be apparent to one of skill in the art.

The fusion protein may be constructed by a variety of mechanisms including, but not limited to, standard DNA manipulation techniques and chemical assembly via subunit parts of the fusion protein. The chemical assembly may lead to an equivalent form as the molecular genetic form or alternative associations with equivalent function. In certain embodiments, the fusion protein is produced by standard recombinant DNA techniques.

The basic principle of the fusion proteins of the present invention is that the distribution of the fusion protein, and the growth factor binding properties associated therewith, are manipulated and directed by the presence of the cell association domain.

Upon binding of the growth factor to the growth factor binding domain of the fusion protein, the ability of the growth factor to stimulate cellular growth or like responses is inhibited, attenuated, or negated. Thus, the distribution of the fusion protein within a subject will typically be determined by the cell association domain where it will influence the growth promoting environment of a cell. Additionally, the cell localization domain may also impart a signal to the cell to slow or stop its growth and/or to undergo apoptosis or other cell death mechanisms.

The exact order of the domains in the fusion protein, as well as the presence and/or length of any other sequences located between or on either end of the domains, is not generally critical, as long as the growth factor binding domain maintains an affinity sufficient to bind the target growth factor and cell association domain maintains an affinity for a target cell type or cell population to affect a localization to that cell type or cell population. Generally, this requires that the two-dimensional and three-dimensional structure of any intervening protein sequence does not preclude the binding or interaction requirements of the domains of the fusion protein. One of skill in the art will readily be able to optimize the fusion protein for these parameters using the teachings herein. An exemplary fusion protein arrangement may be found in SEQ ID NO:16.

For each domain it will be understood that more than one copy of the sequence that imparts or encodes the required function may be present. For example, as used herein, “cell association domain” means an amino acid sequence that imparts a particular distribution to a cell or cell population of the fusion protein. Thus, a first cell association domain and the second cell association domain may each individually comprise 1, 2, or more such amino acid sequences that impart a particular cellular distribution of the fusion protein.

As used herein, “growth factor binding domain” or “ligand binding domain” refers to one or more amino acid sequences to which a growth factor of interest binds. The growth factor binding domain may be a naturally-occurring binding domain, a mutant, variant, or fragment thereof, or an artificial domain. It is to be understood that the growth factor binding domain can comprise a binding site for any growth factor of interest. Thus, the fusion protein of the present invention can bind any type of growth factor that binds to a growth factor binding domain comprising an amino acid sequence. In a preferred embodiment, the binding domain is a binding domain for an insulin-like growth factor.

The growth factor binding domain may comprise of an amino acid sequence for non-covalent binding (such as protein-protein interaction sites), referred to as a “non-covalent binding site,” or an amino acid sequence for binding an subsequently effects an enzymatic reaction, i.e., enzymatic inactivation of a growth factor, referred to as a “covalent binding site.”

In addition, amino acid sequence variants of the polypeptides and/or fusion proteins of the present invention can be substitutional, insertional and/or deletion variants. Deletion variants lack one or more residues of the native protein or a fusion protein that are not essential for a desired function or activity, and are exemplified by the variants lacking amino acid sequences as described below. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue.

Certain insertional mutants and fusion proteins are called chimeras or chimeric proteins. A chimeric protein is a protein in which amino acid sequence segment from one protein that is similar or homologous in function, characteristic or property to an amino acid sequence segment of a second protein are inserted or fused to a second protein in place of the corresponding amino acid sequence segments.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine or histidine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ nucleotide sequences, and yet still be essentially as set forth in one of the sequences disclosed herein, so long as the sequence meets the criteria set forth above, including the maintenance of biological protein activity where protein expression is concerned.

The following is a discussion based upon changing of the amino acids of a protein to create an equivalent, or an improved, second-generation molecule. For example, certain amino acids may be substituted for, inserted in, deleted from, or fused to other amino acids in a protein structure without appreciable loss of interactive binding capacity with structures such as, for example, receptor-binding regions of IGFBPs or peptide-binding region of IGFBPs. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

B. IGFBP Polypeptides

The binding peptides and polypeptides of the invention are typically derived from one or more IGFBPs. In certain embodiments, the present invention concerns novel IGFBP compositions. Embodiments of the invention may comprise all or part of one or more of the polypeptide(s) encoded by SEQ ID NO:1, 3, 5, 7, 9, 11 and/or 13. As used herein, a “proteinaceous molecule,” “proteinaceous composition,” “proteinaceous compound,” “proteinaceous chain” or “proteinaceous material” generally refers, but is not limited to, a protein of greater than about 50 amino acids or the full length sequence translated from a gene, which may encode a fusion between two IGFBPs; a polypeptide of greater than about 100 amino acids; and/or a peptide of from about 5 to about 500 amino acids. All the “proteinaceous” terms described above may be used interchangeably herein.

In certain embodiments the size of the at least one proteinaceous molecule or polypeptide component of a fusion or chimeric protein may comprise, but is not limited to about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, or greater amino acid residues, and any range derivable therein derived from a polypeptide.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, humans, domestic animals or wild animals. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques or the chemical synthesis of proteinaceous materials. The nucleotide, protein, polypeptide and peptide sequences for various IGFBPs have been previously disclosed, and may be found in computerized databases known to those of ordinary skill in the art. One such database is the National Center for Biotechnology Information's Genbank and GenPept databases (www.ncbi.nlm.nih.gov/). For example, exemplary nucleic acid and amino acid sequences for IGFBPs may be found using the following accession numbers: IGFBP-1 accession number NM_{—}000596 (SEQ ID NO:1 and SEQ ID NO:2), IGFBP-2 accession number NM_{—}000597 (SEQ ID NO:3 and SEQ ID NO:4), IGFBP-3 accession number X64875 (SEQ ID NO:5 and SEQ ID NO:6), IGFBP-4 accession number NM_{—}002581 (SEQ ID NO:7 and SEQ ID NO:8), IGFBP-5 accession number NM_{—}000599 (SEQ ID NO:9 and SEQ ID NO:10), IGFBP-6 accession number AJ006952 (SEQ ID NO: 11 and SEQ ID NO:12), or IGFBP-7 accession number NM_{—}001553 (SEQ ID NO:13 and SEQ ID NO:14). The coding regions for known IGFBPs may be amplified and/or expressed using the techniques disclosed herein or as would be know to those of ordinary skill in the art.

In certain embodiments a proteinaceous compound may be purified. Generally, “purified” will refer to a specific IGFBP polypeptide or peptide composition that has been subjected to fractionation to remove various other proteins, polypeptides, or peptides, and which composition substantially retains its activity, as may be assessed, for example, by the protein or binding assays, as would be known to one of ordinary skill in the art.

C. Protein Purification

It may be desirable to purify IGFBP fusion proteins or variants thereof. These techniques involve, at one level, the crude fractionation of the cellular milieu to polypeptide and non-polypeptide fractions.

Fusion or chimeric proteins of the invention may be purified using various detergents known in the art, which include, but are not limited to, NP40 and digitonin. Infected or transfected host cells may be solubilized using a detergent. Conditions such as: 10 mM CHAPS, 0.5% SDS, >2% deoxycholate, or 2.0% octylglucoside may be used. Preparations of substantially nondenatured fusion or chimeric proteins of the invention may be accomplished using techniques described in U.S. Pat. Nos. 6,074,646 and 5,587,285, which are hereby incorporated by reference herein.

Certain aspects of the present invention concern the purification, and in particular embodiments, the substantial purification, of an encoded protein, fusion protein or peptide. The term “purified protein or peptide” as used herein, is intended to refer to a composition, isolatable from other components, wherein the protein, fusion protein or peptide is purified to any degree relative to its naturally-obtainable state. A purified protein, fusion protein or peptide therefore also refers to a protein, fusion protein or peptide, free from the environment in which it may naturally occur.

Generally, “purified” will refer to a protein or peptide composition that has been subjected to fractionation to remove various other components, and which composition substantially retains its biological activity or activities. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein, fusion protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

II. Nucleic Acid Molecules

In some embodiments, the present invention concerns fusion or chimeric proteins prepared from recombinant nucleic acids. Some of the teachings herein pertain to the construction, manipulation, and use of nucleic acids to produce a recombinant fusion or chimeric protein.

A. Polynucleotides Encoding an IGFBP Fusion or Chimeric Protein

The present invention concerns polynucleotides, isolatable from cells, that are free from total genomic DNA and that are capable of expressing all or part of a protein, fusion protein or polypeptide. The polynucleotide may encode a peptide, fusion protein or polypeptide containing all or part of one or more IGFBP amino acid sequence or may encode a peptide, fusion protein or polypeptide having peptide segments derived form two or more IGFBP amino acid sequences. Recombinant proteins can be purified from expressing cells to yield denatured or nondenatured proteins or peptides.

As used herein, the term “DNA segment” refers to a DNA molecule that has been isolated free of total genomic DNA of a particular species. Therefore, a DNA segment encoding a polypeptide or fusion protein refers to a DNA segment that contains wild-type, polymorphic, or engineered polypeptide-coding sequences yet is isolated away from, or purified free from, total mammalian or human genomic DNA. Included within the term “DNA segment” are recombinant vectors, including, for example, plasmids, cosmids, phage, viruses, and the like.

As used in this application, the term “IGFBP fusion or chimeric protein” refers to an engineered IGFBP protein-encoding nucleic acid molecule that has been isolated free of total genomic nucleic acid. Therefore, a “polynucleotide encoding an engineered IGFBP poypeptide” refers to a DNA segment that contains all or part of IGFBP-coding sequences isolated away from, or purified free from, total genomic DNA.

It also is contemplated that a particular polypeptide from a given species may be represented by natural variants that have slightly different nucleic acid sequences but, nonetheless, encode the same protein.

Similarly, a polynucleotide comprising an isolated or purified gene refers to a DNA segment including, in certain aspects, regulatory sequences, isolated substantially away from other naturally occurring genes or protein encoding sequences. In this respect, the term “gene” is used for simplicity to refer to a protein, fusion protein, polypeptide, or peptide-encoding unit. As will be understood by those in the art, this term includes genomic sequences, cDNA sequences, and smaller engineered gene segments that express, or may be adapted to express, proteins, polypeptides, domains, peptides, fusion proteins, chimeric proteins and mutants. A nucleic acid encoding all or part of a native or modified polypeptide may contain a contiguous nucleic acid sequence encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, which may be contiguous nucleotides encoding any length of contiguous amino acids of SEQ ID NO:1, 3, 5, 7, 9, 11, 13, 15 or various combinations of all or part of these sequences.

In particular embodiments, the invention concerns isolated DNA segments and recombinant vectors incorporating DNA sequences that encode an IGFBP fusion or chimeric polypeptide or peptide, such as all or part of IGFBP-6 and/or all or part of IGFBP-3, which includes within its amino acid sequence a contiguous amino acid sequence in accordance with, or essentially corresponding to a native polypeptide(s). Thus, an isolated DNA segment or vector containing a DNA segment may encode, for example, a fusion or chimeric protein that is capable of binding to an IGFBP-3 receptor and/or a growth promoting factor, such as IGF-II. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

Encompassed by certain embodiments of the present invention are DNA segments encoding fusion or chimeric proteins, such as, for example, a peptide comprising all or part of an IGFBP-6 and/or all or part of an IGFBP-3.

In other embodiments, the invention concerns isolated DNA segments and recombinant vectors incorporating DNA sequences that encode a polypeptide, fusion protein or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerable. It is therefore contemplated that a nucleic acid fragment of almost any length may be employed.

It is contemplated that the nucleic acid constructs of the present invention may encode full-length polypeptide, or a combination of two or more polypeptides from any source. A truncated transcript may be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

The DNA segments used in the present invention encompass biologically functional modified polypeptides, fusion or chimeric proteins and peptides. Such sequences may arise as a consequence of codon redundancy and functional equivalency that are known to occur naturally within nucleic acid sequences and the proteins thus encoded. Alternatively, biologically functional proteins, fusion or chimeric proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

The sequence of an IGFBP fusion or chimeric polypeptide will substantially correspond to one or more contiguous portion(s) of the amino acid sequences shown in SEQ ID NO:2, 4, 6, 8, 10, 12, 14, or 16. The term “biologically functional equivalent” is well understood in the art and is defined to include the retention of an ability or function, such as the ability to bind a IGFBP receptor or bind a growth promoting factor.

Accordingly, sequences that have between about 70% and about 80%; or more preferably, between about 81% and about 90%; or even more preferably, between about 91% and about 99%; of amino acids that are identical or functionally equivalent to the amino acids of SEQ ID NO: 2, 4, 6, 8, 10, 12, 14, or 16 will be sequences that are “essentially as set forth in SEQ ID NO: 2, 4, 6, 8, 10, 12, 14, or 16.”

In certain other embodiments, the invention concerns isolated DNA segments and recombinant vectors that include within their sequence a contiguous nucleic acid sequence from that shown in SEQ ID NO:1, 3, 5, 7, 9, 11, 13, or 15. This definition is used in the same sense as described above and means that the nucleic acid sequence substantially corresponds to a contiguous portion of that shown in SEQ ID NO: 1, 3, 5, 7, 9, 11, 13, or 15 and has relatively few codons that are not identical, or functionally equivalent, to the codons of SEQ ID NO: 1, 3, 5, 7, 9, 11, 13, or 15. The term “functionally equivalent codon” is used herein to refer to codons that encode the same amino acid, such as the six codons for arginine or serine, and also refers to codons that encode biologically equivalent amino acids. See Table 2 below, which lists the codons preferred for use in humans, with the codons listed in decreasing order of preference from left to right in the table (Wada et al., 1990). Codon preferences for other organisms also are well known to those of skill in the art (Wada et al., 1990, included herein in its entirety by reference).

The various probes and primers designed around the nucleotide sequences of the present invention may be of any length. By assigning numeric values to a sequence, for example, the first residue is 1, the second residue is 2, etc., an algorithm defining all primers can be proposed:
n to n+y

where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

TABLE 1
PREFERRED HUMAN DNA CODONS

| | |
|---|---|
| Amino Acids | Codons |

| | | | |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

It also will be understood that this invention is not limited to the particular nucleic acid encoding amino acid sequences of SEQ ID NO:2, 4, 6, 8, 10, 12, 14, or 16. Recombinant vectors and isolated DNA segments may therefore variously include IGFBP fusion protein-coding regions themselves, coding regions bearing selected alterations or modifications in the basic coding region, or they may encode larger polypeptides that nevertheless include IGFBP fusion protein-coding regions or may encode biologically functional equivalent proteins or peptides that have variant amino acids sequences.

The DNA segments of the present invention encompass biologically functional equivalent IGFBP fusion or chimeric proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein.

B. Vectors

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (2001) and Ausubel et al., 1996, both incorporated herein by reference. In addition to encoding a modified polypeptide such as IGFBP fusion or chimeric protein, a vector may encode other polypeptide sequences such as a tag or targeting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targeting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

Vectors may include a “promoter,” which is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

D. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC®2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

III. Therapeutic Targets

The present invention deals with the treatment of disease states that involve hyperproliferative disorders including benign and malignant neoplasias. Such disorders include hematological malignancies, restenosis, cancer, multi-drug resistant cancer, psoriasis, inflammatory bowel disease, rheumatoid arthritis, osteoarthritis and metastatic tumors.

In particular, the present invention is directed at the treatment of human cancers including cancers of the prostate, lung, brain, skin, liver, breast, lymphoid system, stomach, testicular, ovarian, pancreatic, bone, bone marrow, head and neck, cervical, esophagus, eye, gall bladder, kidney, adrenal glands, heart, colon, rectum and blood. Other diseases that may be treated with compositions or methods of the invention also may include renal cell carcinomas; viral infections such as, hepatitis C (Garini et al., 2001), HIV-1 (Hatzakis et al., 2001); Erdheim-Chester disease (Esmali et al., 2001), thrombocytopenic purpura (Dikici et al., 2001), marburg hemorrhagic fever (Kolokol'tsov et al., 2001). In certain embodiments, methods and composition are used to treat a subject with neuroblastoma, rhabdomyosarcoma and/or colon cancer In other embodiments, methods and compositions of the invention are used to treat a subject with melanoma.

IV. Combined Therapy

In many therapies, it will be advantageous to provide more than one functional therapeutic. Such “combined” therapies may have particular import in treating multiple aspects of a condition, disease, or other abnormal physiology. For example, treating multidrug resistant (MDR) cancers. Thus, one aspect of the present invention utilizes a modified IGFBP protein comprising a fusion of all or part of two different IGFBPs for the treatment of cancer, while a second therapy, either targeted or non-targeted, is also provided.

A non-targeted treatment may precede or follow modified IGFBP protein treatment by intervals ranging from minutes to weeks. In embodiments where the other agent and modified IGFBP protein are administered separately to the site of interest, one would generally ensure that a significant period of time did not expire between the time of each delivery, such that the agent and the modified IGFBP protein would still be able to exert an advantageously combined effect on a treatment site. In such instances, it is contemplated that one would contact the cell with both modalities within about 12-24 h of each other and, more preferably, within about 6-12 h of each other, with a delay time of only about 12 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the modified IGFBP protein is “A” and the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. For example, in the context of the present invention, it is contemplated that modified IGFBP protein of the present invention could be used in conjunction with non-targeted anti-cancer agents, including chemo- or radiotherapeutic intervention. To kill cells, inhibit cell growth, inhibit metastasis, inhibit angiogenesis or otherwise reverse or reduce the malignant phenotype of tumor cells, using the methods and compositions of the present invention, one would generally contact a “target” cell with a modified IGFBP protein, as described herein and at least one other agent; these compositions would be provided in a combined amount effective achieve these goals. This may be achieved by administering a single composition or pharmacological formulation that includes both agents, or by administering two distinct compositions or formulations, at the same time, wherein one composition includes a modified IGFBP or IGFBP fusion protein, and another includes the other agent.

Agents or factors suitable for use in a combined therapy are any chemical compound or treatment method with therapeutic activity. For example, an “anticancer agent” refers to an agent with anticancer activity. These compounds or methods include alkylating agents, topisomerase I inhibitors, topoisomerase II inhibitors, RNA/DNA antimetabolites, DNA antimetabolites, antimitotic agents, as well as DNA damaging agents, which induce DNA damage when applied to a cell.

Examples of alkylating agents include, inter alia, chloroambucil, cis-platinum, cyclodisone, flurodopan, methyl CCNU, piperazinedione, teroxirone. Topisomerase I inhibitors encompass compounds such as camptothecin and camptothecin derivatives, as well as morpholinodoxorubicin. Doxorubicin, pyrazoloacridine, mitoxantrone, and rubidazone are illustrations of topoisomerase II inhibitors. RNA/DNA antimetabolites include L-alanosine, 5-fluoraouracil, aminopterin derivatives, methotrexate, and pyrazofurin; while the DNA antimetabolite group encompasses, for example, ara-C, guanozole, hydroxyurea, thiopurine. Typical antimitotic agents are colchicine, rhizoxin, taxol, and vinblastine sulfate. Other agents and factors include radiation and waves that induce DNA damage such as, γ-irradiation, X-rays, UV-irradiation, microwaves, electronic emissions, and the like. A variety of anti-cancer agents, also described as “chemotherapeutic agents,” function to induce DNA damage, all of which are intended to be of use in the combined treatment methods disclosed herein. Chemotherapeutic agents contemplated to be of use, include, e.g., adriamycin, bleomycin, 5-fluorouracil (5FU), etoposide (VP-16), camptothecin, actinomycin-D, mitomycin C, cisplatin (CDDP), podophyllotoxin, verapamil, and even hydrogen peroxide. The invention also encompasses the use of a combination of one or more DNA damaging agents, whether radiation-based or actual compounds, such as the use of X-rays with cisplatin or the use of cisplatin with etoposide.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In certain embodiments of the invention localization of a modified IGFBP protein in patients with cancers, precancers, or hyperproliferative conditions will typically be directed to a site of interest by the binding of a portion of the IGFBP fusion protein. Similarly, the chemo- or radiotherapy may be directed to a particular, affected region of a subjects body. Alternatively, systemic delivery of compounds and/or the agents may be appropriate in certain circumstances, for example, where extensive metastasis has occurred.

In addition to combining modified IGFBP protein therapies with chemo- and radiotherapies, it also is contemplated that combination with gene therapies may be advantageous. For example, using a combination of p53, p16, p21, Rb, APC, DCC, NF-1, NF-2, BCRA2, p16, FHIT, WT-1, MEN-I, MEN-II, BRCA1, VHL, FCC, or MCC, or antisense versions of the oncogenes ras, myc, neu, raf, erb, src, fins, jun, trk, ret, gsp, hst, bcl, abl, or any of the genes mentioned above are included within the scope of the invention.

V. Pharmaceutical Compositions

In various embodiments of the invention an IGFBP proteinaceous composition may be administered to a subject as a pharmaceutical composition. The pharmaceutical composition may be used for the treatment of cancers and the like.

Pharmaceutical compositions of the present invention comprise an effective amount of one or more IGFBP proteinaceous composition dissolved or dispersed in a pharmaceutically acceptable carrier or aqueous medium, and may further comprise other anti-bacterial compounds known to one of ordinary skill, including antibiotics or neutralizing antibodies. In particular, Remingtons' Pharmaceutical Sciences, 18^{th} edition (1996), which is incorporated herein by reference, may be consulted for methods of preparation, dosing and the like. The phrases “pharmaceutically or pharmacologically acceptable” refer to molecular entities and compositions that do not produce an adverse, allergic or other untoward reaction when administered to a human, unless specifically designed to elicit such a response. As used herein, “pharmaceutically acceptable carrier” includes any and all solvents, dispersion media, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

These compounds are administered in dosages effective to inhibit, attenuate, or slow the growth of a cancer cell or tumor, where such treatment is needed. Such treatment may also prolong or improve the quality of life of a patient. For use in medicine, the salts of the compounds of this invention refer to non-toxic “pharmaceutically acceptable salts.”

The actual dosage amount of a composition of the present invention administered to a subject can be determined by physical and physiological factors such as body weight and on the route of administration. With these considerations in mind, the dosage of a peptide, polypeptide, polynucleotide, or IGFBP proteinaceous composition for a particular subject and/or course of treatment can readily be determined.

The compositions of the present invention can be administered intravenously, intradermally, intraarterially, intraperitoneally, intraarticularly, intrapleurally, intratracheally, intranasally, intravaginally, topically, intramuscularly, intraperitoneally, subcutaneously, intravesicularlly, mucosally, orally, topically, locally using aerosol, injection, infusion, continuous infusion, localized perfusion bathing target cells directly or via a catheter or lavage. In certain embodiments, such compositions are prepared as injectables, either as liquid solutions or suspensions; solid forms suitable for preparing solutions or suspensions upon the addition of a liquid prior to injection can also be prepared; and the preparations can also be emulsified. The compositions will be sterile, a fluid to the extent that easy syringability exists, stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

Although it is most preferred that compositions of the present invention be prepared in sterile water containing other non-active ingredients, made suitable for injection, solutions of such active ingredients can also be prepared in water suitably mixed with a surfactant, such as hydroxypropylcellulose, if desired. Dispersions can also be prepared in liquid polyethylene glycol and mixtures thereof, and in oils. The carrier can also be a solvent or dispersion medium containing, for example, water, ethanol, polyol (for example, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Upon formulation, solutions will be administered in a manner compatible with the dosage formulation and in such amount as is effective for reducing or inhibiting bacterial colonization or growth in an organism, an organ or a tissue.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Materials: IGF-II and anti IGFBP-6 were purchased from GroPep (Adelaide, Australia) and anti IGFBP-3 was obtained from Upstate Biotechnology (Lake Placid, N.Y.). The IGFBP-3 and IGFBP-6 were produced as previously described (Knudtson et al., 2001).

Cell culture and cell binding: Microvessel endothelial cells were prepared from bovine heart adipose tissue and characterized as previously described (Bar et al., 1986). The neuroblastoma cell lines, SHSY-5Y and SK-NSH, and Rhabdosarcoma lines (RD nd Rh30, were obtained from and grown as recommended by ATCC (Manassas, Va.). or binding studies, iodinated ligand (IGFBP-3, FP 6/3 or IGF-II) (2×10⁴ counts/well) was added to monolayer cultures in 12-well plates either by itself or with unlabelled IGFBPs or fusion protein 6/3. After 90 min to 2 hours at 22° C. the entire monolayer was removed with 0.1 N NaOH and counted in a y counter (Booth et al., 1999).

Thymidine incorporation into DNA: Thymidine incorporation into DNA was performed according to the method of Babajko, et al. (1997) with minor modifications. Cells were plated into 12-well plates, cultured for 3 days in the presence of serum, then changed to serum-free medium (M199+0.25% BSA) for 24 hrs. Cells were used before they reached confluence. After the 24 hr starvation, fresh medium and growth factors/inhibitors were added for up to 18 hrs of incubation. One hour before the end of the incubation 1 μCi/ml of ³H-thymidine (Amersham Biosciences, Piscataway, N.J.) was added. At the end of the incubation the medium was discarded and the cells fixed (5 min) with 5% trichloroacetic acid. After the fixative was removed, cells were digested with 0.1 N NaOH and determination of incorporated radioactivity done in a scintillation counter. Experimental exposure conditions ranged from 10 min to 18 h.

Preparation of FP 6/3: A fusion protein comprised of hIGFBP-6 and hIGFBP-3 was synthesized. The C-terminus of IGFBP-6 was fused to the N-terminus of IGFBP-3 using a clone of hIGFBP-3 and of hIGFBP-6, each having been inserted between the EcoRI and XhoI sites of the cloning vector pSP73. The hIGFBP-6 clone had two of its three existing BsrDI sites silently mutated so that only one near the C-terminus remained. To remove the stop codon and liberate IGFBP-6 from the vector, the IGFBP-6 construct was digested with EcoRI and BsrDI. By digesting the IGFBP-3 construct with SacI and XhoI, IGFBP-3 was released from its signal sequence and pSP73. A sequence to join the two binding proteins was synthesized (Integrated DNA Technologies, Inc., Coralville, Iowa), in the form of complimentary oligonucleotides with sticky ends. The sequence started with nucleotides just after the cut site for BsrDI, encoding the C-terminus of IGFBP-6 (minus the stop codon), then encoding the start of IGFBP-3 through its SacI site. The oligonucleotides were annealed by heating equal molar amounts at 95° C. for 10 min., with a natural cool-down to room temperature. This DNA, along with the IGFBP-6 and IGFBP-3 were directly ligated into the baculovirus expression vector, pBacPAK-9 (BD Biosciences Clontech, Palo Alto, Calif.), between the vector's EcoRI and XhoI sites. The sequence of this new construct was confirmed by DNA sequence analysis. Co-transfection, expression and production of this fusion protein, FP 6/3, was performed as previously described (Knudtson et al., 2001).

Statistical analysis: Data expressed are mean±standard error of means and analyses were by ANOVA/Newman-Keuls. *P<0.05, **P<0.01, ***P<0.001.

### Example 2

### Characterization of the Fusion Protein

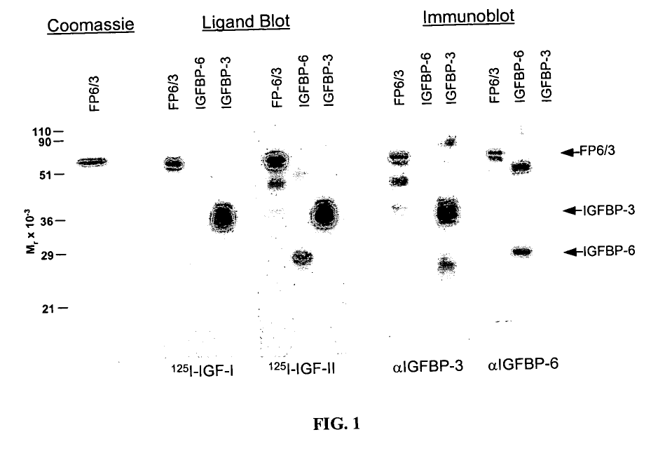

Immunoblotting: FP 6/3 was analyzed by ligand blot and immunoblot (FIG. 1). FP 6/3 was present as one band on Coomassie blue staining and had preferential affinity for IGF-II over IGF-I, as did IGFBP-6.

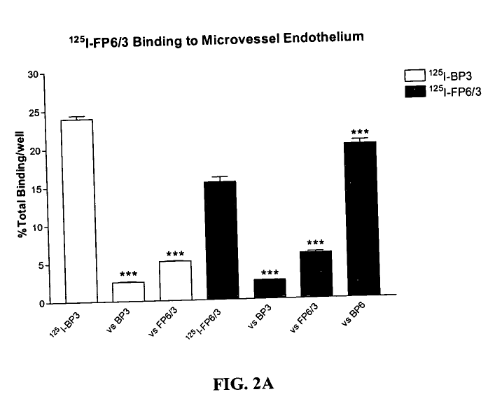

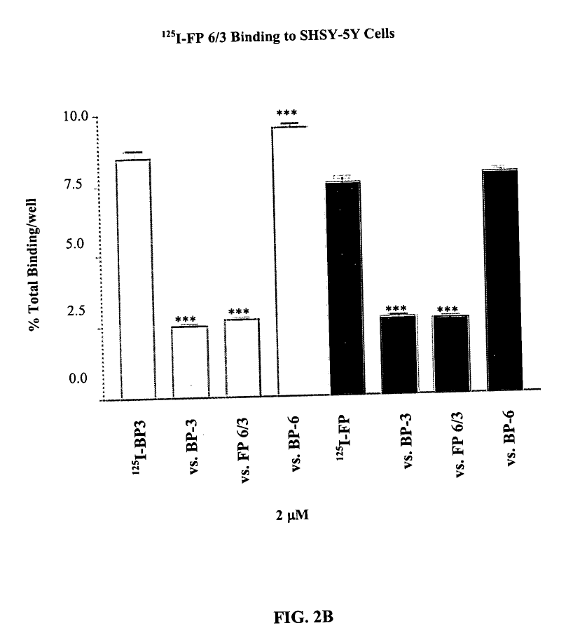

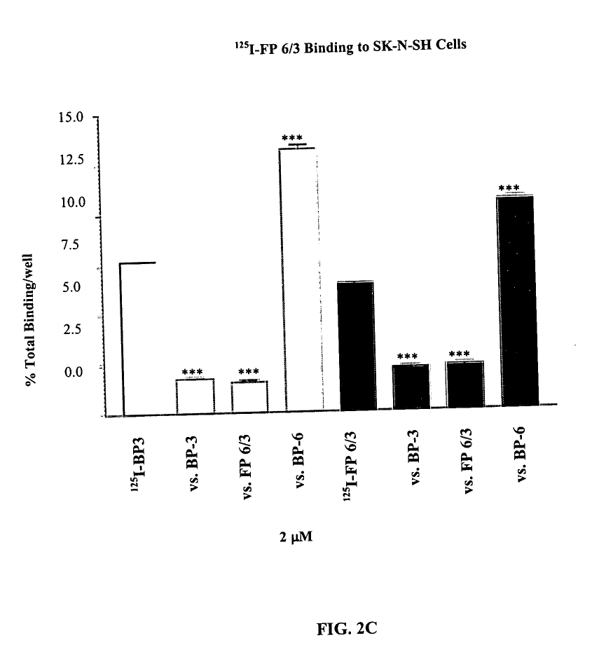

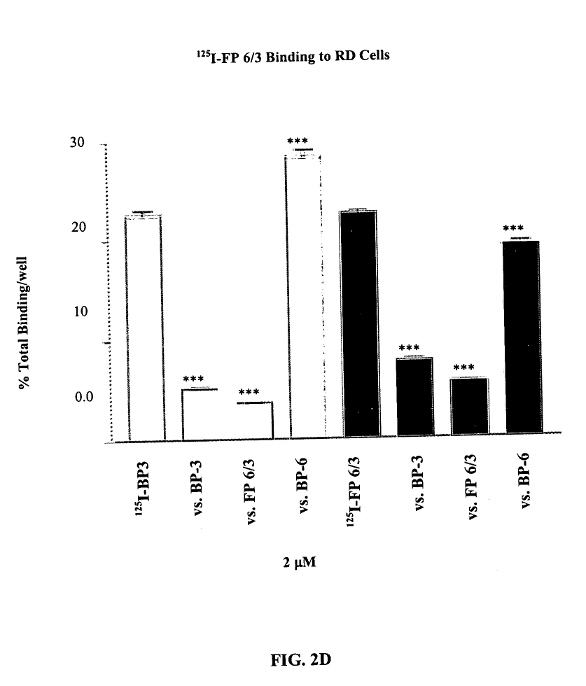

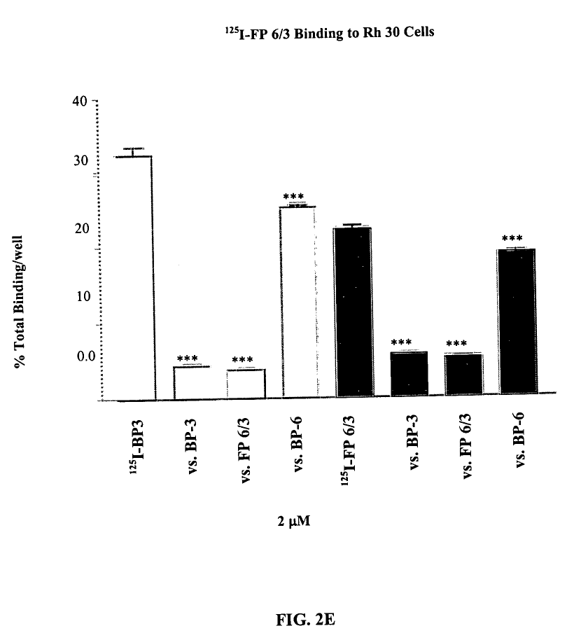

Cell binding: The FP 6/3 was iodinated and specific binding was tested with bovine microvessel endothelial cells, since they have already been proven to have specific binding sites for IGFBP-3 (Hwa et al., 1999). The fusion protein retained its IGFBP-3 characteristic since it was able to bind and displayed its specificity since it was able to be competed by either IGFBP-3 or FP 6/3. The results of binding to microvessel endothelial cells are shown FIG. 2A. IGFBP-6 did not act as a competitor. The ability of the FP 6/3 to specifically bind was also assessed in SHSY-5Y (FIG. 2B), SK-N-SH (FIG. 2C), RD (FIG. 2D) and Rh30 (FIG. 2E) cells yielding similar results. Binding of ¹²⁵I-IGF-II is shown for reference.

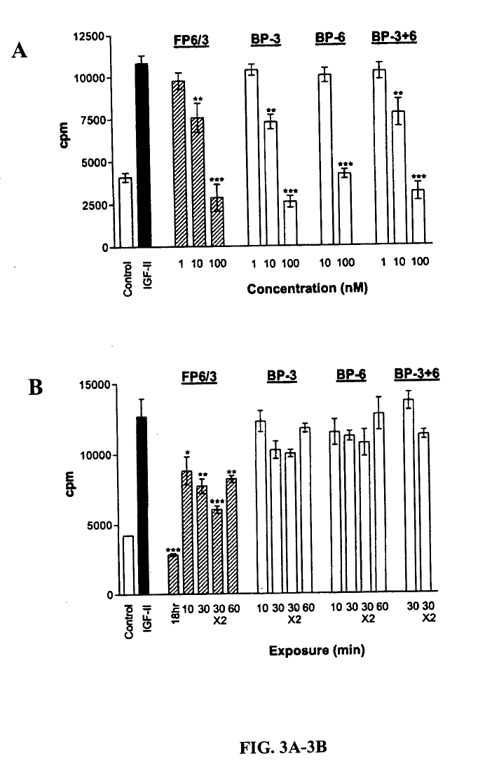

Thymidine Incorporation into DNA: The effects of the fusion protein on thymidine incorporation into DNA were studied in the SHSY-5Y cell line (FIGS. 3A and 3B). IGF-II, a known stimulus to proliferation of SHSY-5Y cells, caused an 3-fold stimulation when given at a concentration of 50 ng/ml. This stimulation could be prevented if the IGF-II was co-incubated with either fusion protein or binding proteins (IGFBP-3, IGFBP-6 or IGFBP-3 plus IGFBP-6) (FIG. 3A). During the co-incubation experiments the maximal concentration of IGF-II was added with increasing amounts (1-100 nM) of fusion protein or binding proteins (FIG. 3A). All caused progressively greater inhibition of thymidine incorporation with each increasing concentration and no significant difference between either FP 6/3 or the IGFBPs was seen (FIG. 3A).

When cells were first exposed to IGFBP-6 or IGFBP-3 for 30 min., then removed and maximal IGF-II stimulus given (6.5 nM), the ability to inhibit IGF-II was not maintained (FIG. 3B). However, by contrast, when cells were initially exposed to the fusion protein for 30 min., then the FP 6/3 removed, the inhibition effect caused by the fusion protein remained even in the presence of maximal IGF-II stimulation (FIG. 3B).

This finding prompted additional studies where transient exposure to FP 6/3 (and to other IGFBPs) was studied under two settings: a) exposing cells to either FP 6/3 or IGFBPs for different durations (10, 30 or 60 min), subsequent removal and final thymidine incorporation measured after 18 h of IGF-II stimulation or b) by treatment frequency where the 30 min exposure was given at time 0 then repeated 9 h later with final thymidine incorporation measured after a total incubation with IGF-II of 18 h (30×2). None of the other binding proteins showed any significant inhibition when transient exposure conditions were used on the neuroblastoma cells even when the frequency was doubled (FIG. 3B). In all transient exposure conditions the concentration of FP 6/3, IGFBP-3, IGFBP-6 or IGFBP-3+IGFBP-6 was 100 nM. Increased frequency of exposure to the FP 6/3 (30×2) produced an additional increase in growth inhibition (P<0.001 vs. IGF-II).

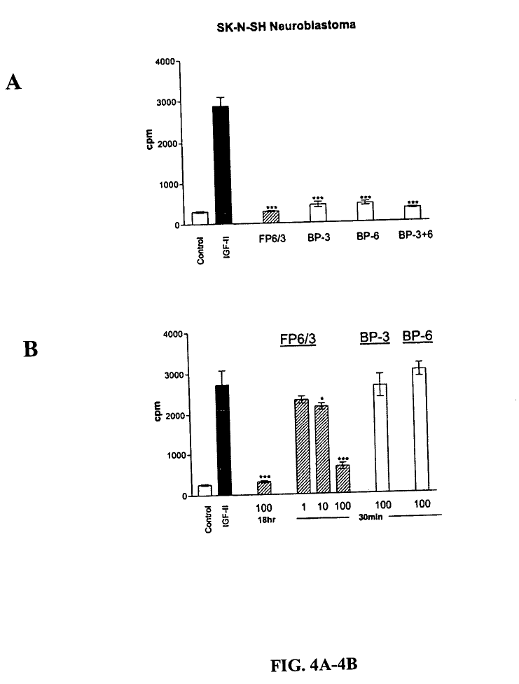

Results in the SK-N-SH cell line were similar to those just described for SHSY-5Y. SK-N-SH cells had specific binding for the fusion protein FP 6/3 (FIG. 2C). The SK-N-SH cells were more responsive to the stimulation of IGF-II (˜9×) on thymidine incorporation into DNA (FIGS. 4A and 4B). They also exhibited growth inhibition when coincubated with FP 6/3, IGFBP-3, IGFBP-6 or IGFBP-3+IGFBP-6 (FIG. 4A). As in the previous study, the transient exposure effect was demonstrated in the SK-N-SH line where exposure to FP 6/3 for only 30 min, then removal, was sufficient to still inhibit thymidine incorporation when tested 18 h later (FIG. 4B). This transient exposure effect was observed even at a concentration of FP 6/3 as low as 1 nM (lowest concentration tested) with a maximal effect at 100 nM (P<0.001 vs. IGF-II). The other binding proteins were unable to inhibit growth when present only transiently (FIG. 4B).

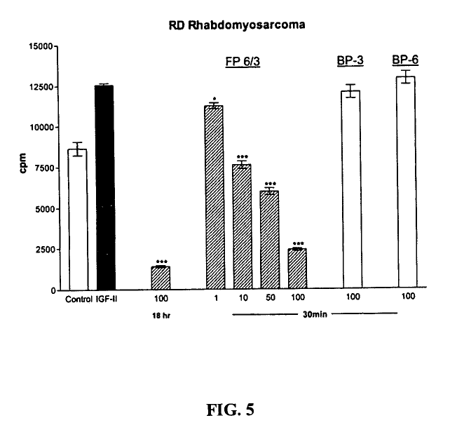

In order to determine that the effect produced by the fusion protein was not limited to just neuroblastoma additional studies were performed using rhabdomyosarcoma (RMS) cell lines. Using the RMS cell lines RD and Rh30, results similar to those just described for neuroblastoma were obtained. Specific binding of ¹²⁵I-FP 6/3 was again demonstrated (FIGS. 2D and 2E). As in the neuroblastoma cell lines, under transient exposure conditions, FP 6/3 was the only binding protein able to produce a growth inhibition effect (FIG. 5). RD control cells displayed a thymidine incorporation level near that of the maximal IGF-II level chosen (6.5 nM) (˜8,600 cpm vs. ˜12,500 cpm) perhaps reflecting a high level of IGF-II produced by these cells. Statistically significant inhibition was observed at a FP 6/3 concentration of 1 nM (P<0.5 vs. IGF-II) underscoring how effective the fusion protein is at neutralizing the IGF-II from both the endogenous as well as exogenous sources even under transient exposure conditions. Progressively greater inhibition was observed as the FP 6/3 concentration was increased with the transient exposure of FP 6/3 at 100 nM (P<0.001 vs. IGF-II) nearly equaling the 18 h exposure of FP 6/3 at 100 nM (P<0.001 vs. IGF-II) (FIG. 5).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,879,236
U.S. Pat. No. 5,587,285
U.S. Pat. No. 5,871,986
U.S. Pat. No. 6,074,646
Allander et al., J. Biol. Chem., 269(14): 10891-10898, 1994.
Ausubel et al., In: Current Protocols in Molecular Biology, John, Wiley & Sons, Inc, New York, 1996.
Babajko et al., J Endocrinology, 152: 221-227, 1997.
Bach, Horm. Metab. Res., 31: 226-234, 1999.
Bar et al., Metabolism, 35: 317-322, 1986.
Baserga, Cancer Res., 55: 249452, 1995.
Baxter et al., J. Clin. Endocrinol. Metab., 65(3): 423-431, 1987.
Booth et al., Growth Regulation, 15: 1-17, 1995.
Buckway et al., J. Clin. Endocrinol. Metab., 86(10): 4943-4950, 2001.
Carbonelli et al., FEMS Microbiol. Lett., 177(1): 75-82, 1999.
Carlsen, Am. J. Pediatr. Hematol. Onc., 14: 103-110, 1992.
Cats et al., Cancer Res., 35 56: 523-526, 1996.
Chan et al., Science, 279: 563-566, 1998.
Chandler et al., Proc. Natl. Acad. Sci. USA, 94(8): 3596-601, 1997.
Cocea, Biotechniques, 23(5): 814-816, 1997.
Cohen and Rosenfeld, Curr. Opin. Pediatr., 6: 462467, 1994.
Cohen et al., J. Clin. Endocrinol. Metab., 75: 1046-1053, 1992.
Cohen, J. Natl. Cancer Inst., 90: 876-879, 1998.
Colao et al., J. Clin. Endocrinol Metab., 84: 1986-1991, 1999.
Deal et al., J. Clin. Endocrinol. Metab., 86(3): 1274-1280, 2001.
Dikici et al., Pediatr. Int., 43(6): 577-580, 2001.
Esmali et al., Am. J. Ophthalmol., 132(6): 945-794, 2001.
Ferry et al., Horm. Metab. Res., 51: 53-67, 1999.
Fraser et al., J. Clin. Endocrinol. Metab., 85(4): 1672-1677, 2000.
Garini et al., Am. J. Kidney Dis., 38(6): E35, 2001.
Giudice, J. Clin. Endocrinol. Metab., 80: 2279-2281, 1995.
Glick et al., J Neurooncol., 35: 315-3125, 1997.
Grellier et al., J. Clin. Endocrinol. Metab., 172: 467-476, 2002.
Grimberg and Cohen, J. Endocrinol. Invest., 22: 64-73, 1999.
Hankinson et al., Lancet., 351: 1393-1396, 1998.
Hatzakis et al., J Interferon Cytokine Res., 21(10): 861-869, 2001.
Holly, Lancet., 351: 1373-1374, 1998.
Hwa et al., Acta Paediatr. Suppl., 88: 37-45, 1999.
Inouye and Inouye, Nucleic Acids Res., 13: 3101-3109, 1985.
Jones and Clemmons, Endocrin. Rev., 16: 3-34, 1995.
Kato, J. Biol. Chem., 270(21): 12373-12379, 1995.
Kelley et al., Intl. Biochem. Cell Biol., 28: 619-637, 1996.
Knudtson et al., Endocrinology, 142: 3749-3755, 2001.
Kolokol'tsov et al., Bull. Exp. Biol. Med., 132(1): 686-688, 2001.
Kostecka and Blahovec, Endocr. Regul., 33(2): 90-94, 1999.
Kyte and Doolittle, J. Mol. Biol., 157(1): 105-132, 1982.
Lahm et al., Int. J. Cancer, 58: 452-459, 1994.
Lamson et al., Growth Factors, 5: 19-28, 1991.
Levenson et al., Hum. Gene Ther., 9(8): 1233-1236, 1998.
Li et al., Cell Tissue Res., 291: 469-479, 1998.
Manousos et al., Int. J. Cancer, 83: 15-17, 1999.
Martin and Baxter, J. Biol. Chem., 274(23): 16407-16411, 1999.
Oh, Breast Cancer Res. Treat., 47: 283-293, 1998.
Orme et al., J. Clin. Endocrinol. Metab., 83: 2730-2734, 1998.
Popovici et al., J. Clin. Endocrinol. Metab., 86(6): 2653-2659, 2001.
Rajah et al., J. Biol. Chem., 272: 12181-12188, 1997.
Rajah et al., Prog. Growth Factor Res., 6: 273-284, 1995.
Rajram et al., Endocrin. Rev., 18: 801-831, 1997.
Rechler, Endocrinology, 138: 2645-47, 1997.
Remington's Pharmaceutical Sciences, 15^{th} ed., 33: 624-652, Mack Publishing Company, Easton, Pa., 1980.
Remington's Pharmaceutical Sciences, 18th Ed. Mack Printing Company, 1996.
Rosen and Pollak, TEM, 10: 136-141, 1999.
Russell et al., Clin. Chem., 44: 705-723, 1998.
Sambrook et al., In: Molecular cloning, Cold Spring Harbor Laboratory Press, Cold Spring Harbor, N.Y., 2001.
Seurin et al., J Cancer (Euro), 38: 2058-2065, 2002.
Shimasaki et al., J. Biol. Chem., 266(16): 10646-10653, 1991.
Spoerri et al., Invest. Ophthalmol. Vis. Sci., 44(1): 365-369, 2003.
Valentinis et al., Mol. Endocrinol., 9: 361-367, 1995.
Wada et al., Nucleic Acids Res., 18: 2367-2411, 1990.
Wolk et al., J. Natl. Cancer Inst., 90: 911-915, 1998.
Yu et al., J. Natl. Cancer Inst., 91: 151-156, 1999.
Zadeh and Binoux, Endocrinology, 138: 3069-3072, 1997.
Zapf et al., J. Clin. Invest., 95(1): 179-186, 1995.

1. A polypeptide comprising all or part of an amino acid sequence of a first IGFBP protein at an amino terminus of the polypeptide and all or part of an amino acid sequence of a second IGFBP at a carboxy terminus of the polypeptide.

2. The polypeptide of claim 1, wherein the polypeptide comprises a growth factor binding domain.

3. The polypeptide of claim 1, wherein the polypeptide comprises a cell association domain.

4. The polypeptide of claim 1, wherein the first IGFBP protein is all or part of IGFBP-6.

5. The polypeptide of claim 4, wherein the first IGFBP protein is IGFBP-6.

6. The polypeptide of claim 1, wherein the second IGFBP protein is all or part of IGFBP-3.

7. The polypeptide of claim 6, wherein the second IGFBP is IGFBP-3.

8. The polypeptide of claim 1, wherein the first IGFBP protein is IGFBP-6 and the second IGFBP protein is IGFBP-3.

9. The polypeptide of claim 1, wherein the polypeptide is comprised in a pharmaceutically acceptable formulation.

10. A nucleic acid comprising a nucleic acid sequence that encodes a polypeptide comprising a fusion of a first IGFBP and a second IGFBP amino acid sequence.

11. The nucleic acid of claim 10, further comprising a promoter region.

12. The nucleic acid of claim 10, further comprising a polyadenylation signal.

13. The nucleic acid of claim 10, wherein the first IGFBP is IGFBP-6.

14. The nucleic acid of claim 10, wherein the second IGFBP is IGFBP-3.

15. The nucleic acid of claim 10, wherein the nucleic acid is comprised in an expression vector.

16. A method of producing a polypeptide comprising:
(a) culturing a host cell comprising a poynucleotide encoding a polypeptide comprising all or part of an amino acid sequence of a first IGFBP protein at an amino terminus of the polypeptide and all or part of an amino acid sequence of a second IGFBP at a carboxy terminus of the polypeptide. under conditions which allow for expression of the polypeptide; and
(b) recovering the polypeptide from the cells.

17. The method of claim 16, wherein the first IGFBP protein is IGFBP-6.

18. The method of claim 16, wherein the second IGFBP protein is IGFBP-3.

19. The method of claim 16, wherein the first IGFBP protien is IGFBP-6 and the second IGFBP protein is IGFBP-3.

20. A method of treatment comprising administering an effective amount of a polypeptide comprising all or part of an amino acid sequence of a first and a second IGFBP protein to a patient having or at risk of developing cancer.

21. The method of claim 20, wherein the first IGFBP protein is IGFBP-6.

22. The method of claim 20, wherein the second IGFBP protein is IGFBP-3.

23. The method of claim 20, wherein the first IGFBP protein is IGFBP-6 and the second IGFBP protein is IGFBP-3.

24. The method of claim 20, wherein the cancer is neuronal, prostate, lung, brain, skin, liver, breast, blood, stomach, testicular, ovarian, pancreatic, bone, bone marrow, head and neck, cervical, esophageal, gall bladder, kidney, adrenal, or colon rectal cancer.

25. The method of claim 20 wherein the cancer is a neuroblasoma or a rhabdomyosarcoma.

26. The method of claim 20, wherein the polypeptide is administered intravenously, intradermally, intraarterially, intraperitoneally, intraarticularly, intrapleurally, intratracheally, intranasally, intravaginally, topically, intramuscularly, subcutaneously, intravesicularlly, mucosally, orally, or by inhallation.

27. The method of claim 20, further comprising administering at least a second anti-cancer therapeutic to the patient.

28. The method of claim 27, wherein the second anti-cancer therapeutic is selected from the group consisting of alkylating agents, topisomerase I inhibitors, topoisomerase II inhibitors, RNA/DNA antimetabolites, DNA antimetabolites, antimitotic agents, and DNA damaging agents.

29. The method of claim 28, wherein the alkylating agent is chloroambucil, cis-platinum, cyclodisone, flurodopan, methyl CCNU, piperazinedione, or teroxirone.

30. The method of claim 28, wherein the topisomerase I inhibitor is camptothecin, camptothecin derivatives, or morpholinodoxorubicin.

31. The method of claim 28, wherein the topoisomerase II inhibitor is doxorubicin, pyrazoloacridine, mitoxantrone, or rubidazone.

32. The method of claim 28, wherein the RNA/DNA antimetabolite is L-alanosine, 5-fluoraouracil, aminopterin derivatives, methotrexate, or pyrazofurin.

33. The method of claim 28, wherein the DNA antimetabolite is ara-C, guanozole, hydroxyurea, or thiopurine.

34. The method of claim 28, wherein the antimitotic agent is colchicine, rhizoxin, taxol, or vinblastine sulfate.

35. The method of claim 28, wherein the DNA damaging agent is γ-irradiation, X-rays, UV-irradiation, microwaves, electronic emissions, adriamycin, bleomycin, 5-fluorouracil (5FU), etoposide (VP-16), camptothecin, actinomycin-D, mitomycin C, cisplatin (CDDP), podophyllotoxin, verapamil, or hydrogen peroxide.

36. The method of claim 27, further comprising administering at least a third anti-cancer therapeutic to the patient.
